(19) United States
(12) Patent Application Publication
(10) Pub. No.: US 2005/0261162 A1
(43) Pub. Date: Nov. 24, 2005

(54) Pharmaceutical composition for preventing and treating cancer and treating an inflammation

(76) Inventors: Kwang-Kyun Park, Seoul (KR); Jae-Kwan Hwang, Gyeonggi-do (KR); Sang-Kook Lee, Seoul (KR); Won-Yoon Chung, Seoul (KR)

Correspondence Address: JONES DAY, 222 EAST 41ST ST, NEW YORK, NY, 10017, US

(21) Appl. No.: 10/472,780

(22) PCT Filed: Mar. 22, 2002
(86) PCT No.: PCT/KR02/00496

§ 371 Date: Sep. 22, 2003

(30) Foreign Application Priority Data
Mar. 22, 2001 (KR) 10-2001-0015027

(51) Int. Cl. A61K 31/00

(52) U.S. Cl. 514/1

(57) ABSTRACT

The present invention relates to a pharmaceutical composition preventing cancer and treating cancer and inflammation, which is characterized in that including xanthorrhizol as an active principle. Xanthorrhizol not only inhibits mutagenesis and tumor formation, and enhances the activity of detoxification enzyme of carcinogen, induces apoptosis of cancer cell, and suppresses the activity of COX-2 and iNOS which are related to tumor promotion and inflammatory reaction. Thus, a pharmaceutical composition including xanthorrhizol can be utilized for prevention of cancer and treatment of cancer and inflammation.

### TECHNICAL FIELD

The present invention relates to a pharmaceutical composition for preventing and treating cancer and treating an inflammation, more particularly, which not only inhibits generation of mutation and tumor, and enhances the activity of detoxification enzyme of carcinogen, and induces apoptosis of cancer cell, but also suppresses the activity of cyclooxygenase-2 (COX-2) and inducible nitric oxide synthase (iNOS) enzyme which are related to the inflammatory reaction.

### BACKGROUND ART

Cancer is now a major worldwide disease which causes 7 million people to die every year, and it was reported that more than about 1.5 million people become new patients suffering from cancer in the United Sates annually in 1997. Considering this tendency, the cancer is assumed to become a leading cause of death before long.

It is known that cancer is caused by various factors. Carcinogens induce mutations by forming adducts to DNA or by bringing about damage to the gene, and it is well-known fact that mutation is a major factor of cancer. Carcinogens are finally converted into ultimate carcinogens by metabolism in the body as well as they flow directly into body.

Carcinogenesis can be classified into the three stages, i.e., initiation, promotion and progression. Initiation begins when DNA in a cell or population of cells is damaged by exposure to exogenous or endogenous carcinogens. If this damage is not repaired, it can lead to genetic mutations. The responsiveness of the mutated cells to their microenvironment can be altered and may give them a growth advantages relative to normal cells. Promotion stage is characterized by selective clonal expansion of the initiated cells, a result of the altered expression of genes whose products are associated with hyperproliferation, tissue remodeling, and inflammation. During tumor progression, preneoplastic cells (benign tumors) develop into malignant tumors through a process of clonal expansion that is facilitated by progressive genomic instability and altered gene expression.

If benign tumors are progressed to malignant tumors, it is irremediable. Therefore, the recent studies are focused on preventing induction, inhibiting or delaying progression of cancers.

Many treatment methods, such as chemotherapy, radiotherapy, surgery therapy and gene therapy, for curing cancer were developed. Among them, chemotherapy by medicine is most commonly used. In former days, the researches to develop the synthetic anti-cancer drugs were performed, but recently, great concerns are concentrated on developing natural materials that are useful for prevention and treatment of cancer.

To develop cancer chemopreventive agents inhibiting tumor formation, National Cancer Institute (NCI) in United State has announced 16 compounds possessing chemopreventive potentials for clinical test referred to Table 1.

TABLE 1

| | | Clinical test | | |
|---|---|---|---|---|
| | Preclinical test | Phase I | Phase II | Phase III |
| 1^{st}Generation | | | | |
| Retinoids | | + | + | |
| Vitamin A | + | | + | + |
| 13-cis-retinoic acid | + | + | + | + |
| 4-HPR | + | + | + | |
| Calcium | + | + | + | |
| β-Carotene | | + | + | + |
| Tamoxifen | + | | | |
| Finasteride | + | | | + |
| 2^{nd}Generation | | | | |
| DFMO | + | + | + | |
| Sulindac | + | | + | |
| Piroxicam | + | + | | |
| Oltipratz | + | + | | |
| N-acetylcysteine | + | + | | |
| Aspirin | + | + | | |
| Ibuprofen | + | + | | |
| Carbenoxole | + | + | | |
| 18-β-Glycyrrhetinic acid | + | + | | |
| DFMO + Piroxicam | + | + | | |
| 3^{rd}Generation | | | | |
| S-Allylcysteine | + | + | | |
| Phenhexyl isothiocyanate | + | | | |
| Curcumin | + | | | |
| Ellagic acid | + | | | |
| Fumaric acid | + | | | |
| Fluasterone | + | | | |
| 4-HPR + Oltipratz | + | | | |
| 4-HPR + Tamoxifen | + | | | |

Among the materials shown at Table 1, curcumin is a pigment component separated from Curcuma longa Linn: (Zingiberaceae) used as a traditional folk medicine in India. It is known that it has excellent anti-oxidant effect and anti-inflammatory effect (Elizabath K. and Rao M. N. A., Int. J. Pharm., 58:237-240, 1990; Tonnesan H. H., Int. J. Pharm., 51: 179-181, 1989), and excellent antimutagenic effect and anticarcinogenic effect and the inhibitory effect on cell proliferation (Nagabhushan M. and Bhide S. V., J. Nutr. Growth Cancer, 4:83-89, 1987; Huang M. T., et al., Cancer Res., 48:5941-5946, 1988; Soudamini K. K. and Kuttan R., J. Ethnopharmacol., 27:227-233, 1989; Jee S. H., et al, J. Invest. Dermatol., 111, 656-661, 1998). Furthermore, it was reported that curcumin suppresses the tumor promotion induced by phobol ester, and shows cytotoxicity against cell lines of human leukemia, colon cancer, CNS, melanoma, kidney cancer and breast cancer (Ramsewak R. S., et al., Phytomedicine, 7:303-308, 2000). NCI has planned a clinical test to develop curcumin to chemopreventive agent (Kelloff G. J., et al., Cancer Epidemiol. Biomarkers Prev., 3:85-98,1994).

Thus, natural products which not only show no side effects and inhibit tumor formation and progression into malignant cancer but also cure inflammation closely related to tumor promotion are continuously being detected.

### DISCLOSURE OF THE INVENTION

The object of the present invention is to provide a pharmaceutical composition not only preventing tumor formation but also treating malignant tumor (cancer) and inflammation by inhibiting mutagenesis and tumor formation by carcinogen, enhancing the activity of enzymes to detoxify carcinogen, inducing apoptosis of cancer cell, and suppressing the activity or expression of COX-2 and iNOS which are closely related to tumor promotion and inflammation.

To achieve the object above-mentioned, the present invention provides a pharmaceutical composition including xanthorrhizol as an effective component for preventing cancer and treating cancer and inflammation.

Xanthorrhizol is a sesquiterpenoid firstly separated from Curcuma xanthorrhiza by Rimpler et al. in 1970, which has a following chemical structure 1.

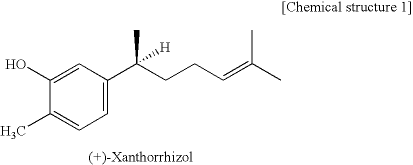

It is reported that xanthorrhizol suppresses the rigid shrinkage of the womb of rat concentration-dependently (Ponce-Monter H., et al., Phytother. Res., 13:202-205, 1999), and shows anti-bacterial activity against oral microorganisms such as Streptococcus mutans (Hwang J. K., Fitoterapia, 71:321-323, 2000; Hwang J. K., Planta Med., 66:196-197, 2000). Said xanthorrhizol could be extracted from Curcuma xanthoffhiza Roxb., a plant of Zingiberaceae family used as an Indonesian folk medicine, and the extraction method such as extraction by organic solvent, extraction by super-critical fluid, microwave extraction and ultrasonic extraction can be used, as disclosed at Korean Patent Laid Open No.2000-73295 and WO 88/05304.

We, the inventors have observed the inhibitory effects of xanthorrhizol on mutagenesis, tumor formation and inflammation. Xanthorrhizol enhanced the activity of carcinogen-detoxifying enzyme, induced apoptosis of cancer cell, inhibited the activity or expression of COX-2 and iNOS which is related to inflammation reaction. Therefore, our results indicate that xanthorrhizol could be effectively used for preventing cancer and treating cancer and inflammation.

The details of the efficacies of preventing cancer and treating cancer and inflammation of xanthorrhizol will be described as follows.

Most of carcinogens are mutagens. Tert-butylhydroperoxide or hydrogen peroxide is known as oxidative mutating agent which result in DNA damage and mutation by generating oxygen radical (Taffe B. G., et al., J. Biol. Chem., 262:12143-12149, 1987; Kappus H., Arch. Toxicol., 60:144-149, 1987), particularly, tert-butylhydroperoxide acts as tumor-promoting agent on mouse skin by forming reactive oxygen species under physiological condition (Epe B., et al., Environ. Health Perspect., 88:111-115, 1990). In the experiments of the present inventors, xanthorrhizol inhibits bacterial mutagenesis induced by tert-butylhydroperoxide or hydrogen peroxide more effectively than curcumin.

Xanthorrhizol effectively inhibits tumor formation in two-stage mouse skin carcinogenesis model (DiGiovanni J., Pharmacol. Ther., 54:63-128, 1992). It suggests that xanthorrhizol is a useful cancer chemopreventive and anticarcinogenic agent.

In addition, xanthorrhizol induces the activation of Phase II detoxification enzyme which suppresses the tumor formation by detoxifying carcinogens in the body. Xanthorrhizol can enhance the ability of body detoxifying carcinogens by activating QR[(NADP(H):quinone oxidoreductase)], a kind of Phase II detoxification enzyme (Talalay P., et al., In: Cancer Biology and Therapeutics. eds. J. G. Cory and A. Szentivanyi. Plenum Press, New York, N.Y., pp. 197-216, 1981). As a result, xanthorrhizol can control the early stage of tumor formation and tumor progression.

The activation of NF-κB increases in tumorigenesis (reference to Cogswell P. C., et al., Oncogene, 19:1123-1131, 2000). The activation of NF-κB is recognized to be critical for regulating the induction of COX-2 and iNOS. One of the critical events in NF-κB activation is dissociation with subsequent degradation of the inhibitory protein IκB via phosphorylation and ubiquitination. Xanthorrhizol can effectively inhibit activation of NF-κB by suppressing degradation of IkBα. It could be understood from above result that xanthorrhizol is a useful agent to inhibit tumor formation.

Xanthorrhizol induces apoptosis of cancer cell. In the process of apoptosis, it is known that the caspase called as interleukin-1β converting enzyme (ICE) plays an important role [Martin, S. J. and Green, D. R., Cell, 82:349-352, 1995]. The caspase group consists of at least 10 caspase enzymes, and has subgroups of ICE(caspase-1,4,5), Ich-1(caspase-2,9), CPP32(caspase-3,6,7,8,10). If the procaspase is activated to a caspase, it activates another caspase which is on the next step, and poly(ADP-ribose)polymerase(PRAP)), a DNA repair enzyme, is decomposed by caspase-3 and activates DNA fragmentation-promoting factor (DFF) to induce apoptosis [Liu X. S., et al., Cell, 89:175-184, 1997]. Morphological characteristics such as DNA fragmentation and nuclear condensation observed commonly at the time of apoptosis show in cancer cells treated with xanthorrhizol.

Xanthorrhizol could be effectively utilized for treatment of inflammation by inhibiting expression of COX-2 and iNOS. It is known that the further each steps of tumorigenesis progresses, the more COX-2 (cycleoxygenase-2) and iNOS (inducible nitric oxide synthase) expression increase (Kitayama W., et al., Carcinogenesis, 20:2305-2310, 1999; Takahashi M., et al., Cancer Res., 57:1233-1237, 1997). Accordingly, it could be understood that there's a close relationship between tumorigenesis and the inflammatory reaction.

Cyclooxygenase (COX) is a key enzyme that catalyzes the biosynthesis of prostaglandins (PGs) from arachidonic acid. Two isoforms of COX, designated COX-1 and COX-2, have been identified. COX-1 is constitutively expressed in most tissues and seems to be responsible for housekeeping roles in normal physiological functions (Amiram R., J.Biol.Chem., 263:3022-2024, 1988). In contrast, COX-2 is not detectible in most normal tissues, but is induced by proinflammatory cytokines, growth factors, oncogenes, carcinogens, and tumor promoters, implying a role for COX-2 in both inflammation and control of cell growth (Subbaramaiah K., Cancer Res., 56:4424-4429, 1996). The increased level of PGs in tumors is due, at least in part, to increased expression of COX-2. Overexpression of COX-2 also inhibits apoptosis and increases the invasiveness of malignant cells (Tsujii M., et al., Proc.Natl.Acad.Sci.USA, 94:3336-3340, 1997). Accordingly, compounds that inhibit selectively the activity or expression of COX-2 might be an important focus for cancer chemoprevention or anti-inflammation.

Nitric oxide synthase (NOS) is another important enzyme involved in regulation of inflammation, vascular tone, neurotransmission, tumor cells and other homeostasis of human body. NOS also exists in the two forms of constitutive form and inducible form. The excessive generation of nitric oxide (NO) is related with pathological vasodilation, cytotoxicity and tissue injury. According to the recent results, NOS increases the permeability of a blood vessel, causes inflammatory reaction such as edema, and promotes the activation of COX to stimulate the biosynthesis of inflammatory mediator such as prostaglandin to induce severe inflammatory reaction. In various cancer tissue, the activation of iNOS is highly increased. Therefore, xanthorrhizol which significantly inhibits the activity of COX-2 and INOS could be utilized not only for prevention of cancer, but also for treatment of inflammation and cancer.

Pharmaceutical composition of the present invention including xanthorrhizol preventing cancer and treating cancer and inflammation could further comprise a pharmaceutically permissible vector and a diluent. Solvent, dispersion medium, absorption retardant and the like which are commercially used in the field of medicine industry can be used as a vector.

Pharmaceutical composition of the present invention for preventing cancer and treating cancer and inflammation could be dosed through whatever general route to reach the target tissue. Therefore, the composition of the present invention could be dosed through an affected part of the body, oral administration, parenteral administration, intra-narial cabity, intravenous injection, intramuscular injection, subcutaneous injection and intrascleral administration. The composition could be formulated as solution, suspended solution, tablet, pill, capsule and sustained releasing agent. The preferred formulation is an injection, and the dosage content of the composition should be determined in consideration of the skill in the art according to the kinds and degree of disease, age, sex and so forth.

### BRIEF DESCRIPTION OF FIGURES

FIG. 1 is a graph representing the inhibitory effect of xanthorrhizol on bacterial mutagenesis induced by tert-butylhydroperoxide(a) and hydrogen peroxide(b).

FIG. 2 is a photograph of agar plate representing the inhibitory effect of xanthorrhizol on mutagenesis induced by hydrogen peroxide.

FIG. 3 is a graph representing the inhibitory effect of the methanol extract of Curcuma xanthorrhiza Roxb(A) and xanthorrhizol(B) against skin tumor formation in two-stage mouse skin carcinogenesis induced by 7,12-dimethylbenz[a]anthracene (DMBA) and 12-O-tetradecanoyl-phorbol-13-acetate (TPA).

FIG. 4 is a photograph of mice showing the inhibitory effect of xanthorrhizol against skin tumor formation in two-stage mouse skin carcinogeneis induced by DMBA and TPA.

FIG. 5 is a graph representing the increase of quinone reductase(QR) activity induced by xanthorrhizol.

FIG. 6 is a western blotting photograph representing that xanthorrhizol inhibits expression of COX-2 protein induced by TPA.

FIG. 7 is a graph representing the inhibitory effect of xanthorrhizol on lipopolysaccharide(LPS)-activated PGE2 production(COX-2 activity).

FIG. 8 is a graph representing the inhibitory effect of xanthorrhizol on LPS-activated nitric oxide production (iNOS activity).

FIG. 9 is a western blotting photograph representing the inhibitory effect of xanthorrhizol on decomposition of IkBα.

FIG. 10 is an agarose gel photograph representing DNA fragmentation induced by xanthorrhizol.

FIG. 11 is a flow cytometric analysis representing the induction of apoptosis by xanthorrhizol.

FIG. 12 is a western blotting photograph representing the activation of procaspase-3 by xanthorrhizol.

### EMBODIMENTS

The more detail description of the present invention is best understood with reference to the preferred embodiments. But the preferred embodiments of the present invention can be variously modified, and the range of the present invention should not be limited to the following embodiments. The embodiments of the present invention are provided for illustrating the present invention more completely to those skilled in the art.

The experimental result is represented as an mean ±SE and IC₅₀, and IC₅₀ is the concentration inhibiting 50% of the reaction. Difference between means of various subgroups is assessed by Student t-test. Statistical significance is defined as a value of P<0.05.

### Example of Separation and Purification of Xanthorrhizol

After extracting the dried rhizome of Curcuma xanthorrhiza with 75% methanol, the extract was fractionated with ethylacetate, butanol, water. A certain single material was purified from ethylacetate fraction by silica gel column chromatography eluted with the mixture of hexane/ethylacetate (10:1, v/v). The purified material was determined to be xanthorrhizol by measuring the molecular weight using EI-MS and by analyzing the ¹H-NMR, ¹³C-NMR and IR spectrum of it.

IR(CDCl₃, Vₘₐₓ) 3402, 2915, 1708, 1620, 1599 cm⁻¹;

EI-MS(m/z) 218, 148, 136, 135, 121; ¹H-NMR(CDCl₃, 400 MHz) 1.18(3H, d, J=7.1 Hz), 1.52(3H, s), 1.57(2H, dt, J=7.1, 7.2 Hz), 1.67 (3H, s), 1.85(2H, dt, J=7.0, 7.2 Hz), 2.20(3H, s), 2.59(1H, qt), 5.08(1H, t, J=7.0, 7.2 Hz), 6.59(1H, br s), 6.66(1H, br d), 7.01(1 H, d, J=7.6 Hz);

¹³C-NMR(CDCl₃, 400 MHz) 147.16, 113.50, 153.51, 120.86, 130.74, 119.42, 38.98, 38.32, 26.10, 124.48, 131.39, 15.31, 25.67, 17.64, 22.3

### Embodiment 1

### The Antimutagenic Effect on Mutagenesis Induced by Reactive Oxygen Species

The antimutagenic effect of xanthorrhizol was examined in Salmonella typhimurium TA102 strain including mutagenesis with reactive oxygen species (Levin, D. E., et al., Proc. Natl. Acad. Sci. U. S. A., 79;7445-7449, 1982).

Salmonella typhimurium TA102 strain was cultured in Oxoid nutrient broth medium for 11 hours. 100 μl of above-cultured medium was added to 600 μl of the reaction mixture containing tert-butylhydroperoxide (100 μg/plate) or hydrogen peroxide (50 μg/plate) with or without xanthorrhizol and incubated for 30 minutes at 37° C. Curcumin was added instead of xanthorrhizol in positive control. The concentration of xanthorrhizol or curcumin was 0, 10, 20, 40, 60 nmol/plate and 2, 4, 8, 10, 20, 50 nmol/plate respectively in experiment to examine the inhibitory effect of xanthorrhizol against tert-butylhydroperoxide and hydrogen peroxide-induced mutagenesis. The reaction mixture was transferred to 2 ml of top agar solution containing 0.5 mM of histidine and biotin and was homogeneously mixed. It was poured to minimal glucose plate. The plates were incubated for 48 hours at 37° C. and the number of His+ revertant colonies counted.

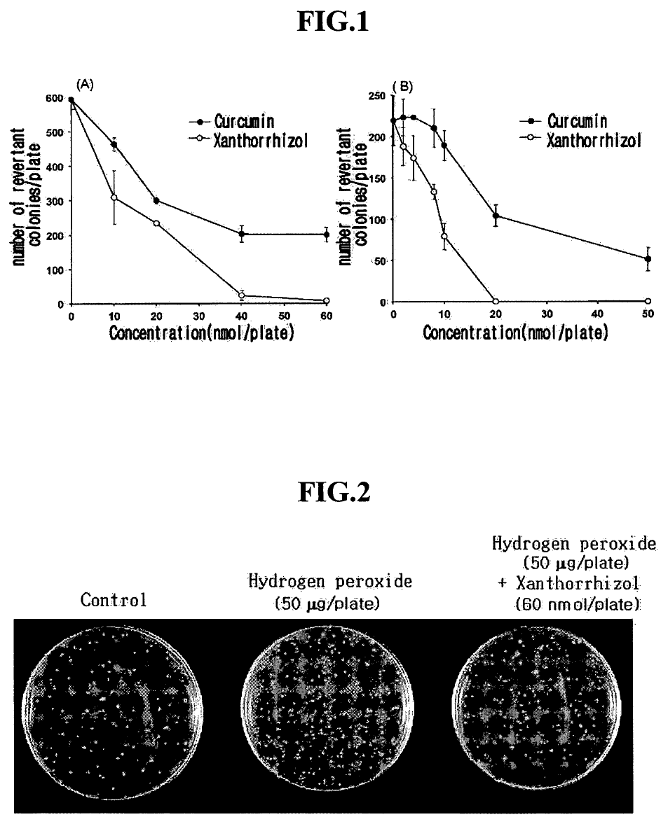

The antimutagenic effect against mutagenesis induced by tert-butylhydroperoxide(a) and hydrogen peroxide(b) was represented at graph (A) and (B) at FIG. 1, respectively, and a photograph of agar plate representing the antimutagenic effect of xanthorrhizol against mutagenesis induced by hydrogen peroxide are shown at FIG. 2. As shown in FIG. 2, xanthorrhizol showed more excellent inhibitory effect against mutagenesis induced by tert-butylhydroperoxide and hydrogen peroxide than curcumin used as a positive control.

### Embodiment 2

### The Inhibitory Effect on Tumor Formation in Two-Stage Mouse Skin Carcinogenesis Model

The chemoprotective effect of xanthorrhizol and the methanolic extract of Curcuma xanthorrhiza Roxb. against tumor formation was investigated in multistage mouse carcinogenesis induced by tumor initiator (DMBA) and tumor promoter (TPA).

The methanolic extract of Curcuma xanthorrhiza Roxb. was prepared as follows. After cutting the dried Curcuma xanthorrhiza into small pieces, 400 ml of 75% methanol was added to 100 g of the sample and extracted repeatedly for 2 days at room temperature. The methanolic extract was filtered with Whatman filter paper, evaporated and dried by freeze-drier.

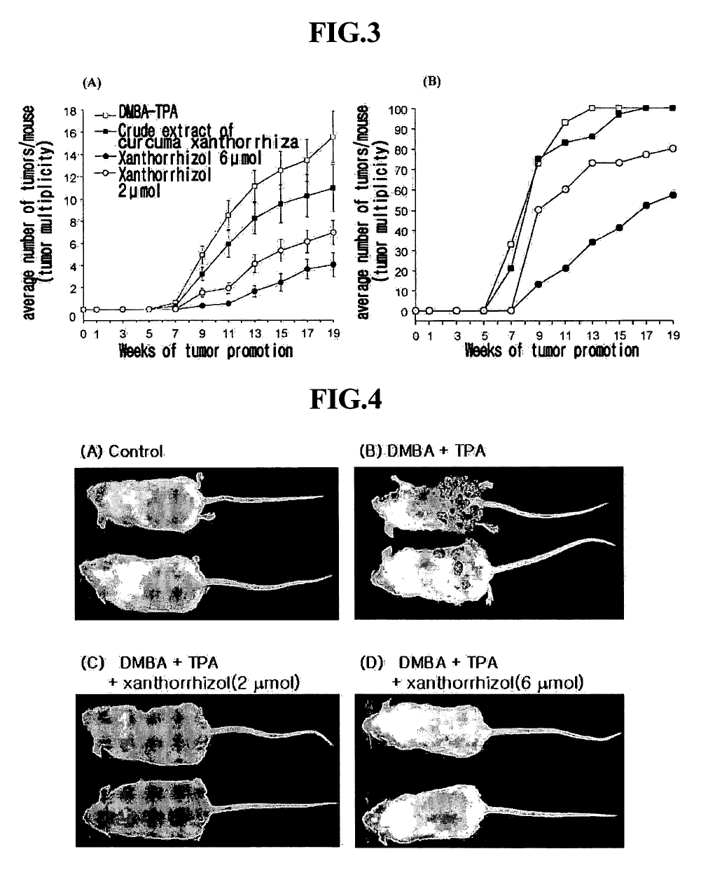

To evaluate the inhibitory effect of xanthorrhizol and the methanolic extract of Curcuma xanthoffhiza Roxb. against tumor formation, 30 mice (6 weeks age, female) per an experimental group was used. The dorsal region of ICR mice was shaved with an electric clipper. After a topical application of 0.2 μmol DMBA in 0.2 ml acetone, mice were treated topically with xanthorrhizol or the methanolic extract of Curcuma xanthorrhiza 30 min prior to each topical application of 10 nmol TPA in 0.2 ml acetone which was continued three times weekly for 19 weeks. The negative control was treated with only 0.2 ml acetone. Tumors were counted and recorded biweekly. The results were expressed as the average number of tumors per mouse (tumor multiplicity) and the percentage of tumor-bearing mice (tumor incidence) and are shown at FIG. 3 and FIG. 4. The graph (A) of FIG. 3 represents the tumor multiplicity of each experimental group and the graph (B) shows the tumor incidence. FIG. 4 is a photograph representing the inhibitory effect of xanthorrhizol against tumor formation at 19 weeks.

As shown in FIG. 3 and FIG. 4, xanthorrhizol inhibits tumor formation dose-dependently. All of the mice treated with DMBA and TPA without xanthorrhizol had tumors with an average of 15.5 skin tumors. On the other hand, mice given topical application of 6 μmol xanthorrhizol three times per week for 19 weeks developed an average of 4.0 skin tumors per mouse and 57% of the treated mice had tumors. These results indicate that xanthorrhizol is an excellent chemopreventive agent reducing tumor incidence and tumor multiplicity significantly.

### Embodiment 3

### Induction of Quinone Reductase Activity

Hepa 1c1c7 cell (2.5×10⁴/ml), a liver cancer cell of rat, was seeded into 96 well plate and was cultured in 10% FBS-αL-MEM (Gibco BRL) at 37° C. for 24 hours in 5% CO₂ of humidified air. 190 μl of fresh media and 10 μl of xanthorrhizol dissolved in 10% of DMSO was added to above culture media and it was cultured under 5% CO₂ at 37° C. for 48 hours. The culture media were discarded, and after washing with PBS (phosphate buffered saline), 50 μl of reaction solution containing 0.8% digitonin and 2 mM EDTA was added to each well, and it was cultured for 10 minutes to destroy the cell. After the plate was shaken in the orbital shaker (100 rpm) for 10 minutes, 200 μl of reaction solution containing menadione and MTT(3-[4,5-dimethylthiazol-2-yl]-2,5-diphenyl-tetrazolium bromide) (final reaction solution 50 ml: 2.5 mL of 0.5M-tris, 0.34 ml of 1.5% Tween-20, 0.034 ml of 7.5 mM FAD, 0.334 ml of 150 mM G-6-P, 30 μl of 50 mM NADP, 100 μl of Glucose 6-phosphate dehydrogenase, 33.4 mg of BSA, 15 mg of MTT, 50 μl of 50 mM menadione) was added to react for 10 minutes. Then, 50 μl of 5 mM potassium phosphate (pH 7.4) solution containing 0.3 mM of dicoumarol was added to terminate the reaction and the absorbance at 595 nm was measured spectrophotometrically.

To assess the effect of xanthorrhizol on cell growth, protein was measured in the 2^{nd} plate set cultured under same condition above. After removing the culture media, the cell was treated with 0.2% crystal violet for 10 minutes, then washed with tap water and dried. And 200 μl of 0.5% SDS was added to cell and mixed, then the absorbance at 595 nm was measured spectophotometrically.

To estimate the experimental result, firstly, QR specific activity of each group treated with xanthorrhizol and the control group was calculated by following equation 1. The relative level of QR activity induced by xanthorrhizol, that is, QR induction ratio (treated/control) was defined as the ratio between QR specific activity of the group treated with xanthorrhizol and that of control by following equation 2. The concentrations of xanthorrhizol used were 50, 10, 2, 0.4 μM, respectively.
QR specific activity=(Absorbance change of MTT per min/Absorbance change of crystal violet)×3247 nmol/mg [Equation 1]
QR induction ratio=Specific activity of test sample treated with xanthorrhizol/Specific activity of control [Equation 2]

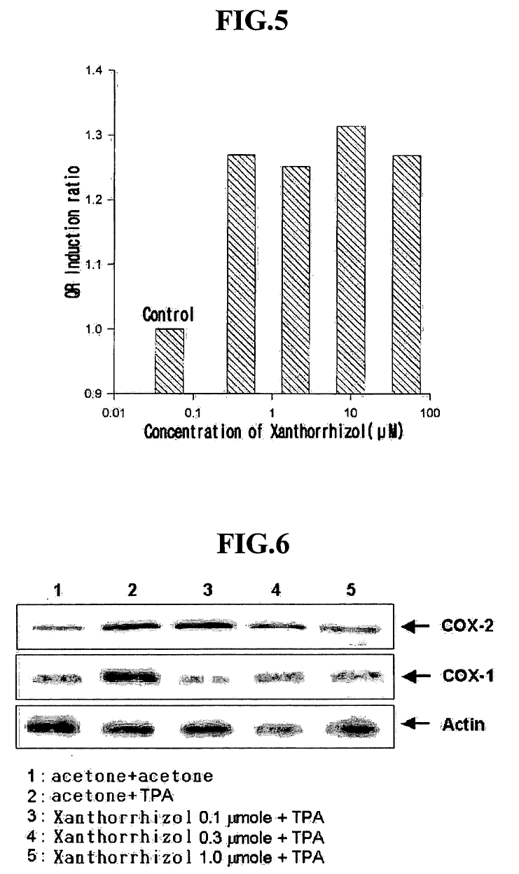

QR induction ratio by xanthorrhizol represented at FIG. 5. As shown in FIG. 5, QR induction ratio at 0.4 μM and 50 μM of xanthorrhizol is about 125% and 130%, respectively, compared with the control. These results suggest that xanthorrhizol could contribute to removal of carcinogen in the body by increasing the activity of enzyme detoxifying carcinogens such as QR.

### Embodiment 4

### Inhibition of COX-2 Expression Induced by TPA

It is known that the expression of COX-2 increases in mouse skin treated with TPA. Therefore, the effect of xanthorrhizol on COX-2 expression induced by TPA was measured as follows on the basis of this fact.

Female ICR mice of about 5 weeks of age were purchased from the Daehan Experimental Animal Center (Seoul, Korea). Mice were kept on a 12 h light/dark cycle.

The dorsal region of mice was shaved with an electric clipper. 2 days later, xanthorrhizol dissolved in 0.2 ml acetone was topically applied on mouse skin followed by topical application of TPA (10 nmol) dissolved in 0.2 ml acetone after 30 min. Mice were sacrificed by cervical dislocation 4 hr later. The skin was excised and the fat was removed. Fat-free skin was immediately placed in liquid nitrogen and pulverized in mortar.

Pulverized mouse skin was lysed in 400 μl of lysis buffer [150 mM NaCl, 0.5% Triton X-100, 50 mM tris-HCl, pH 7.4, 20 mM EGTA, 1 mM DTT, 1 mM Na₃VO₄, protease inhibitor cocktail tablet] for 30 min on ice. Lysates were centrifuged and total protein in supernatant was quantified by Bio-Rad protein assay. Aliquots of supernatant containing 30 μg protein were boiled in SDS sample loading buffer for 5 min before electrophoresis on a 12% SDS-polyacrylamide gel. Blots were transferred from SDA-polyacrylamide gel to PVDF membrane, blocked with 5% fat-free dry milk-PBS buffer containing 0.1% Tween 20 (PBST) for 2 hr at room temperature and then washed with PBST buffer. Membranes were incubated for 1 hr at room temperature with goat COX-2 polyclonal antibody for 2 hr. Blots were rinsed with PBST, incubated with anti-goat horseradish peroxidase-conjugated secondary antibody (Zymed Laboratories Inc., San Francisco, Calif., USA) and then washed again 3 times in PBST buffer for 5 min. Transferred proteins were visualized with an ECL (Enhanced chemiluminescence) detection kit. Western blotting of COX-2 was shown in FIG. 6. Referring FIG. 6, the expression of COX-2 induced by TPA was decreased by pretreatment with xanthorrhizol in a dose-dependent manner.

### Embodiment 5

### Inhibition of COX-2 Activity Induced by Lipopolysaccharide (LPS)

If a cell was treated with LPS, the activity of COX-2 increases. On the basis of this fact, to investigate the effect of xanthorrhizol on LPS-induced COX-2 activity, the quantity of PGE₂ released from cells was measured as follows.

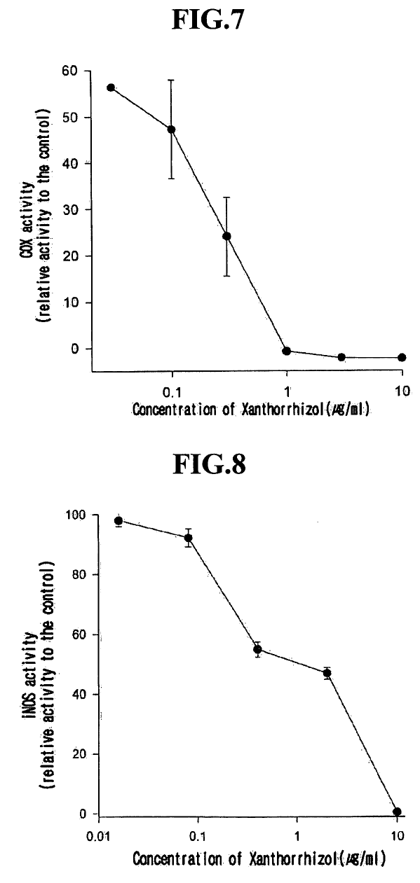

RAW264.7 macrophage cells were maintained in DMEM supplemented with penicillin-streptomycin and 10% FBS at 37° C., in 5% CO₂ of humidified air. The cells (10×10⁵ cells/ml, 200 μl) were allowed to adhere for 4 hr in the presence of aspirin (500 μM) in a 96-well culture plate to inhibit irreversibly COX activity in cells, washed 3 times with media, and then incubated in the fresh medium with 1 μg/ml of LPS. Xanthorrhizol was simultaneously added to each well. After an additional 16 hr incubation, the media were recovered and analyzed by PGE₂ enzyme immunometric assay. The medium recovered from each well was added to each well attached anti-PGE₂ antibody (Amersham Life Science, Arlington Heights, Ill.) with PGE₂-acetylcholineesterase tracer, incubated for 18 hr at room temperature and then washed five times with 0.05% Tween 20-phosphate buffer solution. 200 μl of Ellman reagent was added to each well and incubated for 7 hr. Absorbance at 405 nm was measured. PGE₂ in each medium treated with xanthorrhizol was quantified in calibration curve graphed with standard PGE₂. 100% activity is defined as the difference between PGE₂ accumulation in the absence and in the presence of LPS for 16 hr in triplicate determinations. The percentage inhibition was expressed as [1-(PGE₂ level of sample/PGE₂ level of vehicle treated−control)]×100. The result is shown in FIG. 7.

FIG. 7 demonstrates that xanthorrhizol inhibits the activity of COX-2 induced by LPS dose-dependently, especially xanthorrhizol shows not less than 98% of percentage inhibition (IC₅₀=0.07 μg/ml=0.32 μM) at the concentration of not less than 1 μg/ml. This result suggests that xanthorrhizol can inhibit inflammation and tumor promotion by blocking COX-2 activity.

### Embodiment 6

### Inhibition of iNOS Activity Induced by LPS

The effect of xanthorrhizol on iNOS activity induced by LPS was measured. RAW264.7 macrophage cells were maintained in DMEM supplemented with penicillin-streptomycin and 10% FBS at 37° C., in 5% CO₂ of humidified air. The cells in 10% FBS-DMEM without phenol red media were plated in 24-well plates (8×10⁵/ml), and then incubated for 4 hr. The cells were replaced with new media, and incubated in the medium with 1 μg/ml of LPS and xanthorrhizol. After an additional 20 hr incubation, the media were removed and analyzed for nitrite accumulation as an indicator of NO production by the Griess reaction. 150 μl of Griess reagent were added to 100 μl of each supernatant from LPS and/or xanthorrhizol treated cells in triplicate. The plates were incubated for 10 min, and were read at 570 nm against a standard curve of NaNO₂. The percentage inhibition was expressed as [1−(NO level of sample/NO level of vehicle treated−control)]×100. The result is shown in FIG. 8.

Referring to FIG. 8, xanthorrhizol inhibits dose-dependently the activity of iNOS induced by LPS, and particularly shows not less than 99% of percentage inhibition at the concentration of 10 μg/ml (IC₅₀=1.01 μg/ml=4.63 μM). This result suggests that xanthorrhizol can mitigate inflammation and tumor promotion by inhibiting production of nitric oxide.

### Embodiment 7

### Inhibition of lκB Degradation in Mouse Skin Treated with TPA

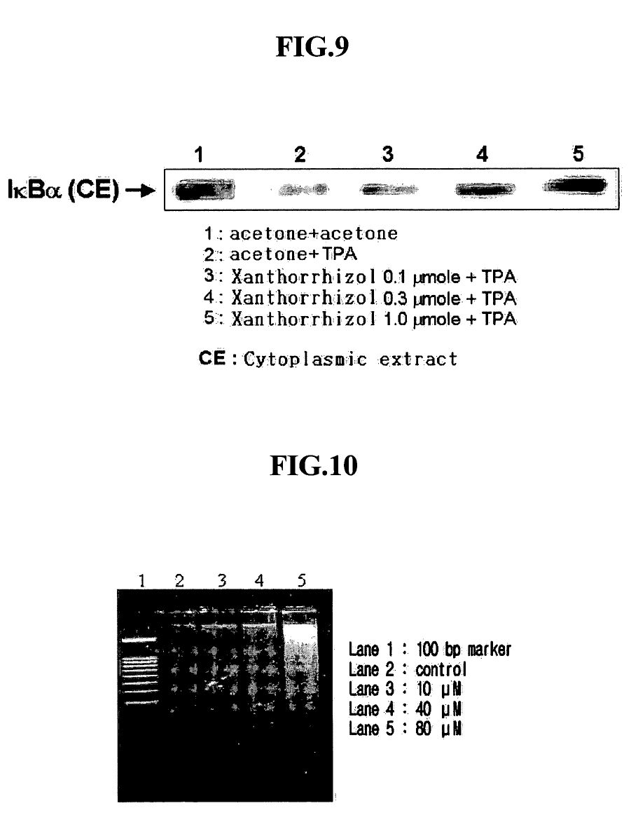

To examine the effect of xanthorrhizol on IκB, the level of IκB was measured in mouse skin. Cytoplasmic extract was prepared as follows. The mouse skin tissue obtained by the same method of embodiment 4 was homogenized in hypotonic buffer solution [10 mM HEPES, pH 7.8, 10 mM KCl, 2 mM MgCl₂, 1 mM DTT, 0.1 mM EDTA, 0.1 mM phenylmethylsulfonyl fluoride (PMSF)]. To the homogenates was added 125 μl of 10% Nondiet P-40 solution and the mixture was then centrifuged for 30 sec. The supernatant (cytoplasmic extract) was electrophoresized on the 12% SDS-polyacrylamide gel. Blot was transferred from SDS-polyacrylamide gel to PVDF membrane, blocked with 5% fat-free dry milk-PBST buffer for 2 hr at room temperature and then washed in PBST buffer. Membrane was incubated for 2 hr at room temperature with rabbit IκBα polyclonal antibody (Santa Cruz Product, Santa Cruz, Calif., USA). Blot was rinsed with PBST, incubated with anti-rabbit horseradish peroxidase-conjugated secondary antibody (Santa Crus product, Santa Cruz, Calif., USA) and again washed 3 times in PBST buffer for 5 min. Transferred protein was visualized with an ECL detection kit. The western blotting photograph was shown in FIG. 9. Referring FIG. 9, it could be understood that the degradation of IκBα induced by TPA is inhibited by xanthorrhizol in a dose dependent manner.

### Embodiment 8

### Induction of Apoptosis by Xanthorrhizol

Human promyelocytic leukemia (HL-60) cells were maintained at 37° C. in a humidified atmosphere of 95% air and 5% CO₂ in RPMI 1640 supplemented with 10% (v/v) heat-inactivated fetal bovine serum (FBS). HL-60 cells were cultured in 6-well plate in RPMI 1640 medium containing 10% FBS in the absence or presence of the methanolic extract of Curcuma xanthorrhiza (15 μg/ml) and xanthorrhizol (40 μM) and centrifuged after 24 hr. 4% neutral buffered formaline was added to the cell and the mixture was transferred to slides, which were left at room temperature for dryness. The fixed cells were washed in PBS, air-dried and stained with DNA-specific fluorochrome Hoechest 33258 for 1 min. The adhered cells were washed with PBS, air dried, and mounted with 50% glycerol. The slides were observed by fluorescence microscopy. The result showed morphological characteristics of apoptosis such as distinct chromatin condensation and nuclear fragmentation in HL-60 cells treated by Curcuma xanthorrhiza and xanthorrhizol.

HL-60 cells were cultured in 10% FBS-RPMI 1640 medium of 100 mm Petri dish for 2 days. The cells were treated with 0,10, 40, 80 μM of xanthorrhizol to investigate the effect of xanthorrhizol on DNA fragmentation, a biochemical marker of apoptosis. After 24 hr, the cells were collected, incubated with 500 μl of lysis buffer (1 % Triton-X 100, 50 mM Tris-HCl pH 7.4, 20 mM EDTA) for 1 hr on ice, and centrifuged. To the supernatant was added 100 μl of 1% SDS, 10 μl f TE/RNase(10 mg/ml), 50 pl of proteinase K (1 mg/ml) and the mixture was incubated at 37° C. at least for 4 hr. DNA was extracted with phenol-chloroform-isoamylalcohol (25:24:1, v/v) and precipitated at −70° C. for 1 hr after addition of 2.5 volumes of cold ethanol. DNA fragments were resolved by 1.5% agarose gel electrophoresis and visualized by staining with ethidium bromide. The result of electrophoresis is shown in FIG. 10, demonstrating that DNA fragmentation, a biochemical marker of apoptosis, was induced by 80 μM of xanthorrhizol.

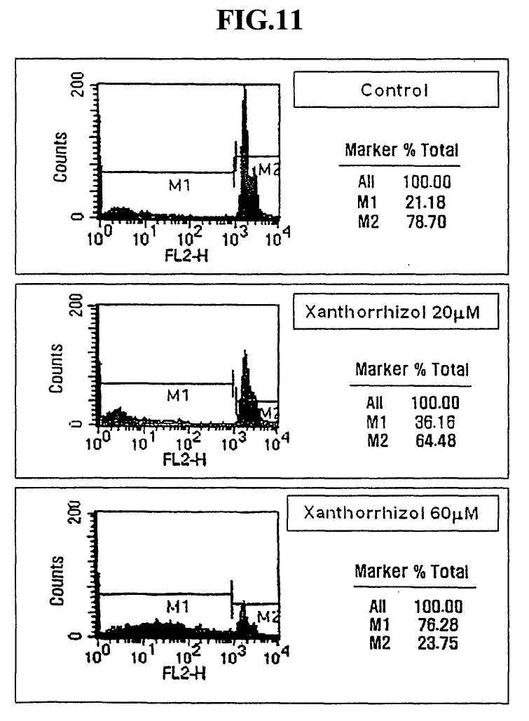

The effect of xanthorrhizol on cell cycle was examined by flow cytometric analysis. HL-60 cells were cultured in serum-free RPMI 1640 medium for 48 hr to stop cell cycle at GO phase. The medium was exchanged to 10% FBS-RPMI 1640 media with 0, 20, 60 μM of xanthorrhizol, respectively. 24 hours later, the cells obtained after centrifugation were fixed in 70% ethanol at −20° C. overnight. The cells were washed twice again with PBS, and incubated with 100 U/ml of Rnase at 37° C. for 1 hr. The cell pellet was resuspended in propidium iodide solution after washing twice with PBS. The cells were analyzed by flow cytometry and the result was represented at FIG. 11.

As shown in FIG. 11, 20% in control and 36% and 76% in cells treated with 20 μM and 60 μM of xanthorrhizol respectively were the proportions of cells in sub-G1 phase compartments [apoptosis peak, M1 fraction, sub-diploid DNA content]. This result shows that xanthorrhizol induces apoptosis concentration-dependently.

### Embodiment 9

### Activation of Procaspase-3 by Xanthorrhizol

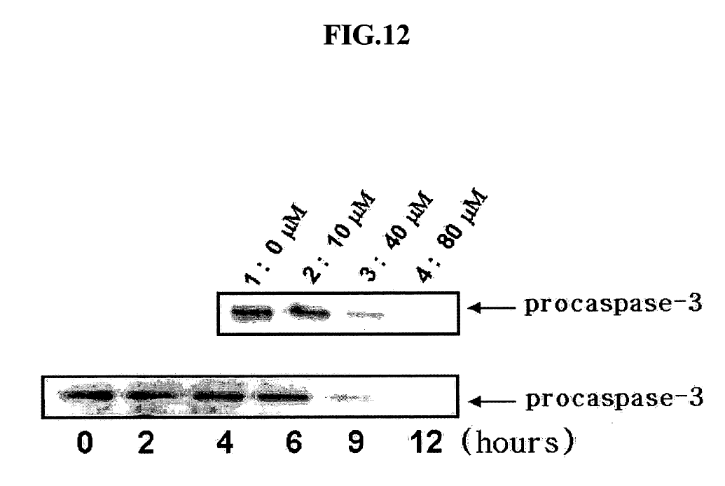

To investigate whether xanthorrhizol also induces the activation of procaspase-3, HL-60 cells were treated with 0, 10, 40, 80 μM of xanthorrhizol for 24 hr and was also treated with 80 μM of xanthorrhizol for 0, 2, 4, 6, 9 and 12 hr. The cells were harvested, suspended in 400 μl of lysis buffer described in embodiment 4, incubated 4° C. for 40 min and centrifuged. The supernatant was electrophoresized on the 12% SDS-polyacrylamide gel. Blot was transferred from SDS-polyacrylamide gel to PVDF membrane, blocked with 5% fat-free dry milk-PBST buffer for 2 hr at room temperature and then washed in PBST buffer. Membrane was incubated for 2 hr at room temperature with mouse procaspase-3 monoclonal antibody (Transduction Laboratories, Lexington, Ky., USA). Blot was rinsed with PBST, incubated with mouse horseradish peroxidase-conjugated secondary antibody and again washed 3 times in PBST buffer for 5 min. Transferred protein was visualized with an ECL detection kit. The western blotting photograph of procaspase-3 is shown in FIG. 12.

Referring FIG. 12, 40 μM of xanthorrhizol activated the procaspase-3 to caspase-3.

Taken together, xanthorrhizol inhibits bacterial mutagenesis and mouse skin formation, enhances the activity of carcinogen-detoxifying enzyme, induces apoptosis of cancer cell and suppresses significantly the activity and expression of COX-2 and iNOS which are closely related to tumor promotion as well as inflammation. Therefore, a pharmaceutical composition including xanthorrhizol is very useful for prevention of cancer and treatment of cancer and inflammation.

1-2. (canceled)

3. A method of treating or preventing cancer in a human or animal which comprises administering to the human or animal in need thereof an effective amount of xanthorrhizol.

4. A method of treating or preventing inflammation in a human or animal comprising administering to the human or animal in need thereof an effective amount of xanthorrhizol.

5. The method of claim 3 or 4, wherein the administration is oral or parenteral.

6. The method of claim 3 or 4, wherein the administration is rectal, vaginal, topical or transdermal.

7. The method of claim 5, wherein the administration is intravenous, intramuscular, intraperitoneal, or subcutaneous.

8. A pharmaceutical composition suitable for treating or preventing cancer or inflammation comprising an effective amount of xanthorrhizol and a pharmaceutically acceptable carrier, diluent or excipient.

9. The pharmaceutical composition of claim 8, wherein the pharmaceutical composition is adapted for oral or parenteral administration to a patient.

10. The pharmaceutical composition of claim 8, wherein the pharmaceutical composition is adapted for rectal, vaginal, topical or transdermal administration to a patient.

11. The pharmaceutical composition of claim 9, wherein the administration is intravenous, intramuscular, intraperitoneal or subcutaneous.

12. A single unit dosage form which comprises the pharmaceutical composition of claim 8.

13. The dosage form of claim 12 wherein the dosage form is adapted for oral or parenteral administration to a patient.

14. The dosage form of claim 12 wherein the dosage form is adapted for rectal, vaginal, topical or transdermal administration to a patient.

15. The pharmaceutical composition of claim 13, wherein the administration is intravenous, intramuscular, intraperitoneal or subcutaneous.
